(19) United States
(12) Patent Application Publication
(10) Pub. No.: US 2024/0342398 A1
(43) Pub. Date: Oct. 17, 2024

(54) NEBULIZER

(71) Applicant: Murata Manufacturing Co., Ltd., Kyoto (JP)

(72) Inventors: Kenghua HSING, Kyoto (JP); Miki IKEDA, Kyoto (JP); Hiroaki WADA, Kyoto (JP); Yohei KAWASAKI, Kyoto (JP); Susumu TAKEUCHI, Kyoto (JP)

(21) Appl. No.: 18/753,520

(22) Filed: Jun. 25, 2024

Related U.S. Application Data
(63) Continuation of application No. PCT/JP2023/002298, filed on Jan. 25, 2023.

(30) Foreign Application Priority Data
Jan. 26, 2022 (JP) 2022-010229

(51) Int. Cl. A61M 11/00

(52) U.S. Cl. CPC A61M 11/005; A61M 2205/0294

(57) ABSTRACT

A nebulizer includes a case and a nozzle. The case has an inner wall which defines an internal space. The nozzle atomizes a liquid stored in the internal space. The case includes a discharge outlet for guiding the atomized liquid to the outside of the case. The nozzle has a gas hole and a liquid hole. The gas hole ejects a gas. The liquid hole is located adjacent to the gas hole and ejects the liquid. In the internal space, the case does not have any path formed of a straight line which extends from the gas hole to the discharge outlet by passing only through the internal space.

### CROSS REFERENCE TO RELATED APPLICATION

This is a continuation of International Application No. PCT/JP2023/002298 filed on Jan. 25, 2023 which claims priority from Japanese Patent Application No. 2022-010229 filed on Jan. 26, 2022. The contents of these applications are incorporated herein by reference in their entireties.

### BACKGROUND OF THE DISCLOSURE

### Field of the Disclosure

The present disclosure relates to a nebulizer.

### Description of the Related Art

Patent Document 1 discloses a nebulizer. The nebulizer includes a case having an internal space and a nozzle that atomizes a liquid. The case has a discharge outlet for guiding the atomized liquid to the outside of the case. The nozzle is located in the internal space of the case. The nozzle has a gas hole and a liquid hole. The gas hole is used for ejecting an output gas. The liquid hole is used for ejecting a liquid.
Patent Document 1: Japanese Unexamined Patent Application Publication No. 2015-159991

### BRIEF SUMMARY OF THE DISCLOSURE

In a nebulizer, such as that disclosed in Patent Document 1, as a path for atomized droplets, a path formed by a straight line extending from the liquid hole to the discharge outlet of the case can be used. Droplets traveling along this path include large droplets which are not sufficiently atomized. Hence, in a nebulizer, such as that disclosed in Patent Document 1, large droplets which are not sufficiently atomized as intended may be outputted to the outside of the case.

To solve the above-described problem, an aspect of the disclosure provides a nebulizer including a case and a nozzle. The case has an inner wall which defines an internal space. The nozzle is located in the internal space and atomizes a liquid stored in the internal space. The case has a discharge outlet for guiding the atomized liquid to outside of the case. The nozzle has a gas hole and a liquid hole. The gas hole ejects a gas. The liquid hole is located adjacent to the gas hole and ejects the liquid. In the internal space, the case does not have any path formed of a straight line which extends from the gas hole to the discharge outlet by passing only through the internal space. A point is specified on the inner wall of the case and is set to a specific point. The inner wall of the case has at least one specific point which is specified on the inner wall. The specific point is a point from which a virtual line segment that links the gas hole and the specific point by passing only through the internal space and a virtual line segment that links the specific point and the discharge outlet by passing only through the internal space are able to be drawn.

With the above-described configuration, large droplets can be prevented from traveling straight and reaching the discharge outlet. It is thus possible to prevent large droplets which are not atomized sufficiently from being outputted to the outside of the case.

With the above-described configuration, a flow path of a gas from the gas hole to the discharge outlet is curved at one portion. Among droplets traveling along a flow of the gas, large droplets are unable to turn at the curved corner of the flow path and adhere to the inner wall of the case. On the other hand, small droplets traveling along the flow path of the gas are likely to smoothly travel in accordance with the flow of the gas within the case because of their lightweight. That is, the small droplets are most likely to reach the discharge outlet without colliding with the inner wall of the case. Since the flow path of the gas is curved only at one portion, small droplets do not excessively adhere to the inner wall of the case. As a result, small droplets, which are sufficiently atomized, can be outputted to the outside of the case at a sufficiently high flow rate while large droplets are being removed.

It can make it less likely to output large droplets to the outside of a case.

### BRIEF DESCRIPTION OF THE SEVERAL VIEWS OF THE DRAWINGS

FIG. 1 is a perspective view of a nebulizer of an embodiment.

FIG. 2 is an exploded perspective view of the nebulizer of the embodiment.

FIG. 3 is a perspective view of a nozzle and a tank in the embodiment.

FIG. 4 is a sectional view of the nebulizer of the embodiment.

FIG. 5 is a sectional view of a nebulizer of a modified example.

FIG. 6 is a sectional view of a nebulizer of another modified example.

### DETAILED DESCRIPTION OF THE DISCLOSURE

### First Embodiment

A first embodiment of a nebulizer will be described below with reference to the drawings. For easy understanding of the embodiment, elements may be shown in enlarged size in the drawings. The dimensional ratio of elements in one drawing may be different from that in another drawing or that of actual elements.

### (Overall Configuration)

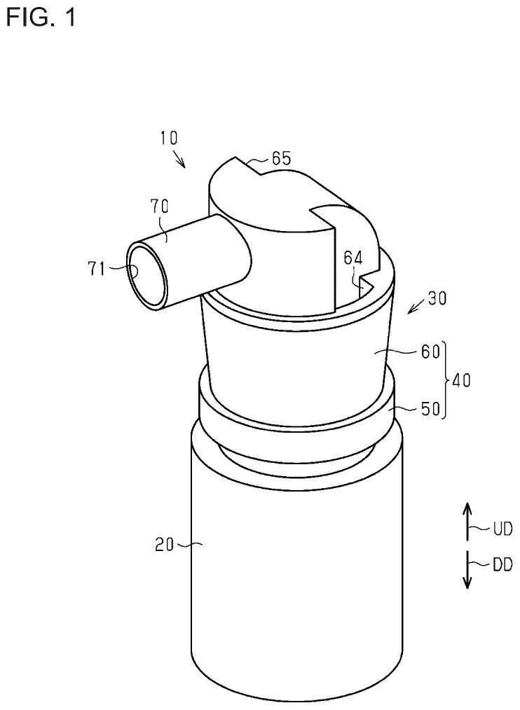

As illustrated in FIG. 1, a nebulizer 10 includes a pump case 20 and a case 30. A gas pumped out of the pump case 20 is supplied to an internal space S of the case 30.

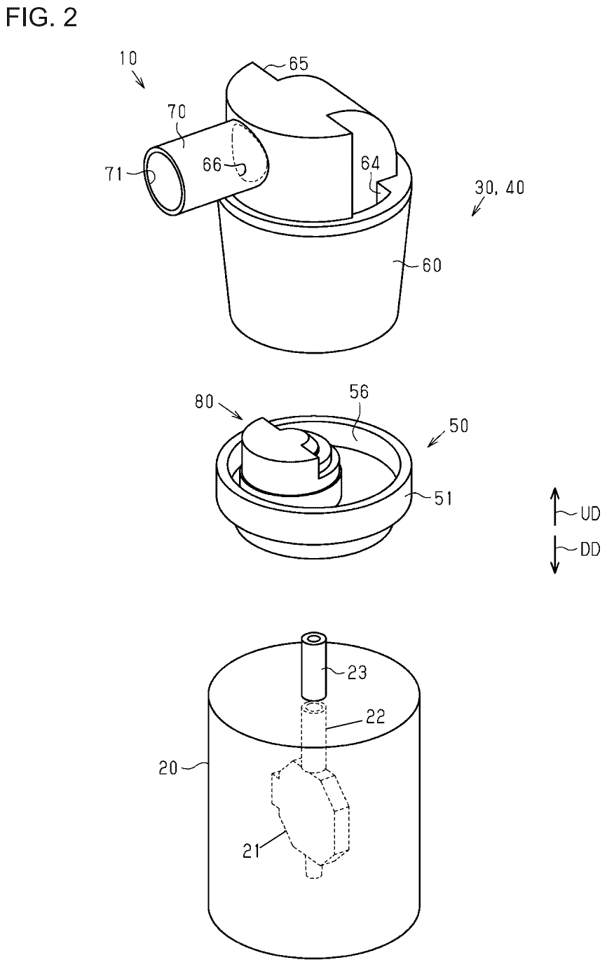

As illustrated in FIG. 2, the nebulizer 10 includes a pump 21 and a tube 22. The pump 21 is located inside the pump case 20. The pump 21 is what is known as a piezoelectric pump. The pump 21 includes a piezoelectric element and a diaphragm. The piezoelectric element is a piezoelectric ceramic element. The diaphragm repeatedly deflects due to the vibration of the piezoelectric element so that the pump 21 can supply air. That is, the pump 21, which is a piezoelectric pump, can output a gas.

The tube 22 is connected to the pump 21. Air pumped out of the pump 21 flows inside the tube 22. The pump case 20 includes a connecting pipe 23. The connecting pipe 23 has a tubular shape. The connecting pipe 23 is linked with the tube 22 so that air pumped out of the pump 21 flows out of the connecting pipe 23 of the pump case 20.

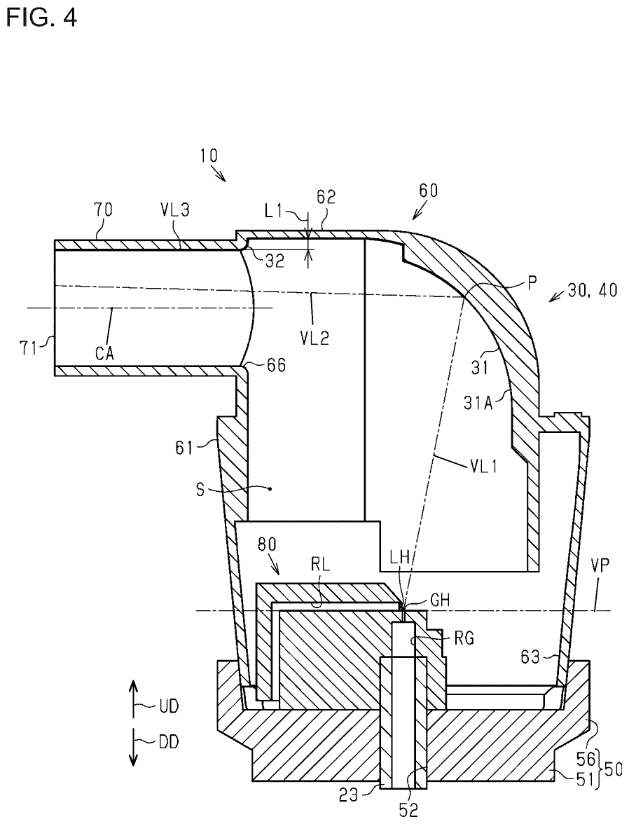

As shown in FIG. 4, the case 30 has an inner wall 31 that defines the internal space S. The case 30 also has a discharge outlet 71 for guiding a liquid atomized in the internal space S to the outside of the case 30. Details of the case 30 will be discussed later.

As shown in FIG. 2, the nebulizer 10 includes a nozzle 80. As illustrated in FIG. 4, the nozzle 80 is located in the internal space S of the case 30. The nozzle 80 atomizes a liquid stored in the internal space S.

### (Nozzle)

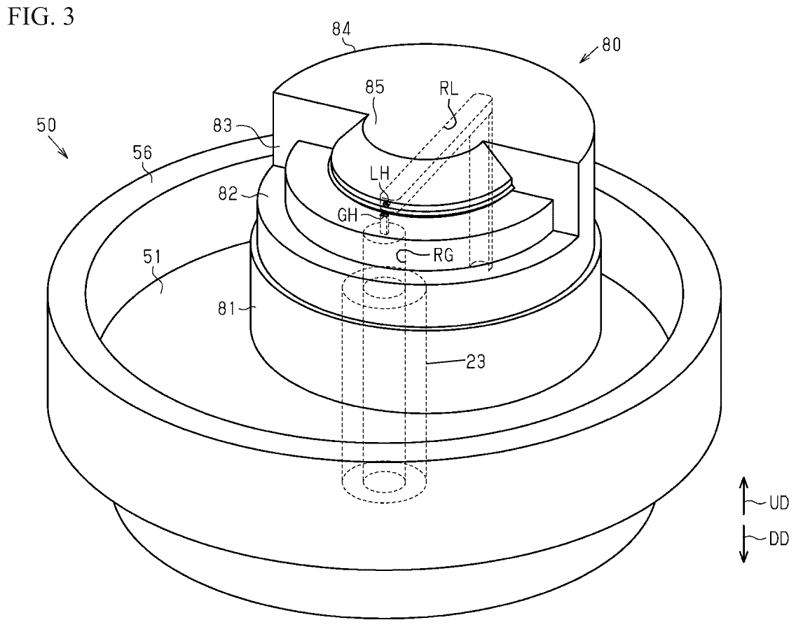

As shown in FIG. 3, the nozzle 80 has a gas supply path RG that links the internal space S and the inside of the connecting pipe 23 with each other. The gas supply path RG is a space for causing a gas pumped out of the pump 21 to flow to the internal space S via the connecting pipe 23. The nozzle 80 has a gas hole GH, which is positioned at an end of the gas supply path RG closer to the internal space S.

The pointing direction of the gas hole GH is set to a first direction. In the embodiment, the first direction is called an upward direction UD, and the opposite direction of the upward direction UD is called a downward direction DD. The pointing direction of the gas hole GH is a direction determined in the following manner. A viewpoint from which the apparent area of a range surrounded by the outer periphery of the gas hole GH is maximized is first specified. Then, the outer periphery of the gas hole GH is seen from this viewpoint and the centroid of the outer periphery of the gas hole GH is specified. Then, the direction from the specified centroid to the viewpoint is set to the pointing direction of the gas hole GH.

The nozzle 80 has a liquid supply path RL through which a liquid stored in the internal space S is supplied to the vicinity of the gas hole GH. The nozzle 80 has a liquid hole LH, which is an opening of the liquid supply path RL in the upward direction UD. The liquid hole LH is located adjacent to the gas hole GH, so that a liquid ejected from the liquid hole LH is supplied toward the gas hole GH. A state in which a liquid is discharged from the liquid hole LH due to a flow of a gas ejected from the gas hole GH is assumed as a state in which the gas hole GH and the liquid hole LH are adjacent to each other.

As illustrated in FIG. 3, the nozzle 80 includes a first portion 81 and a second portion 82. The first portion 81 has a cylindrical shape. The second portion 82 has a cylindrical shape. The second portion 82 is disposed on the surface of the first portion 81 which faces the upward direction UD.

The nozzle 80 also includes a third portion 83. The third portion 83 is disposed on the surface of the second portion 82 which faces the upward direction UD. The third portion 83 has a planar shape. When the nozzle 80 is seen from above in the downward direction DD, the third portion 83 covers a semicircular section of the second portion 82 and the center of the second portion 82 and its vicinity. Accordingly, part of the surface of the second portion 82 which faces the upward direction UD is not covered with the third portion 83, that is, it is projected from the third portion 83 and is exposed.

The nozzle 80 also includes a fourth portion 84. The fourth portion 84 is disposed on the surface of the third portion 83 which faces the upward direction UD. The fourth portion 84 has a semicircular planar shape in a plan view. When the nozzle 80 is seen from above in the downward direction DD, the position of the center of the assumed circle of the semicircular fourth portion 84 coincides with that of the center of the circle of the first portion 81. When the nozzle 80 is seen from above in the downward direction DD, the fourth portion 84 covers a semicircular section of the third portion 83.

The nozzle 80 also includes a fifth portion 85. The fifth portion 85 is disposed on the surface of the third portion 83 which faces the upward direction UD. The fifth portion 85 has a semicircular planar shape in a plan view. When the nozzle 80 is seen from above in the downward direction DD, the position of the center of the assumed circle of the semicircular fifth portion 85 coincides with that of the center of the circle of the first portion 81. The diameter of the semicircle of the fifth portion 85 is shorter than that of the fourth portion 84. The fifth portion 85 covers part of the surface of the third portion 83 which faces the upward direction UD. Accordingly, part of the surface of the third portion 83 which faces the upward direction UD is covered with neither the fourth portion 84 nor the fifth portion 85, that is, it is projected from the fourth and fifth portions 84 and 85 and is exposed.

The gas hole GH is opened to the surface of the third portion 83 which faces the upward direction UD. The gas hole GH is located at a portion of this surface which is projected from the fourth and fifth portions 84 and 85. The gas hole GH is located near the side surface of the fifth portion 85.

The liquid hole LH is opened to the side surface of the fifth portion 85. The liquid hole LH is positioned in the vicinity of the gas hole GH. As stated above, the liquid hole LH is located adjacent to the gas hole GH. A liquid discharged from the liquid hole LH is thus supplied toward the gas hole GH.

### (Case)

As illustrated in FIG. 1, the case 30 includes a case body 40 and a pipe 70. The case body 40 houses the nozzle 80 therein. The case body 40 includes a tank 50 and a cover 60.

As illustrated in FIG. 2, the tank 50 has a bottom wall 51 and a side wall 56. The bottom wall 51 has a disc-like shape. As shown in FIG. 4, the bottom wall 51 has a connecting hole 52 whose flow path has a circular cross section. The connecting hole 52 passes through the bottom wall 51. The connecting pipe 23 of the pump case 20 is fitted in the connecting hole 52.

As illustrated in FIG. 2, the side wall 56 protrudes from the outer periphery of the bottom wall 51 toward the opposite direction of the pump 21. The side wall 56 extends from the entirety of the outer periphery of the bottom wall 51. That is, the side wall 56 has a generally tubular shape.

As shown in FIG. 4, the cover 60 has a side wall 61 having a tubular shape as a whole and a top wall 62. The top wall 62 covers the opening of the side wall 61 which faces the upward direction UD. The opening of the side wall 61 which faces the downward direction DD serves as a connection port 63 having a substantially circular shape. The diameter of the connection port 63 substantially coincides with that of the outer periphery of the side wall 56 of the tank 50 which is on the opposite side of the bottom surface 51. The connection port 63 of the cover 60 is connected to the periphery of the side wall 56 of the tank 50 which faces the upward direction UD.

As illustrated in FIG. 2, the cover 60 has a first intake hole 64 and a second intake hole 65. Both of the first and second intake holes 64 and 65 link an internal space and an external space of the cover 60 with each other in the up-down direction.

The cover 60 has an exit 66 that links the internal space and the external space of the cover 60 with each other. The exit 66 is positioned farther in the downward direction DD than the top edge of the side wall 61 of the cover 60. The exit 66 has a circular shape. The pointing direction of the exit 66 is the opposite direction of the pointing direction of the liquid hole LH.

The pipe 70 has a tubular shape. A first end of the pipe 70 is linked to the case body 40. More specifically, the first end of the pipe 70 is connected to the exit 66 of the cover 60. The pipe 70 extends in the pointing direction of the exit 66. That is, the pipe 70 extends in a direction perpendicular to the upward direction UD.

As shown in FIG. 4, a virtual plane VP perpendicular to the upward direction UD is imagined. In this case, a central axis CA of the pipe 70 extends in parallel with the virtual plane VP.

The pipe 70 has the discharge outlet 71, which is an opening of the pipe 70 on the opposite side of the cover 60. The discharge outlet 71 is an opening for guiding an atomized liquid to the outside of the case 30. That is, a second end of the pipe 70 serves as the discharge outlet 71. The internal space S is defined by the inner wall 31 of the case 30 including the inner wall of the tank 50, the inner wall of the cover 60, and the inner wall of the pipe 70. When a user uses the nebulizer 10, part of the nebulizer 10 including the discharge outlet 71 of the pipe 70 is inserted into the mouth of the user.

Part of the inner wall 31 of the case 30, which faces the exit 66, is formed as a curved surface 31A. The curved surface 31A is a concave surface. In other words, the curved surface 31A is a convex surface projecting to the outside of the case 30.

### (Details of Internal Space)

A path from the gas hole GH to the discharge outlet 71 in the internal space S of the case 30 will be explained below.

In the case 30, virtual straight line segments which link the gas hole GH and the discharge outlet 71 are drawn. In this case, all the virtual line segments intersect with the inner wall 31 of the case 30. Accordingly, within the internal space S, the case 30 does not have any path formed of a straight line which extends from the gas hole GH to the discharge outlet 71 by passing only through the internal space S without intersecting with the inner wall 31 of the case 30.

As illustrated in FIG. 4, a point specified on the inner wall 31 of the case 30 is set to a specific point P. In this case, in the internal space S, the following first virtual line segment VL1 and second virtual line segment VL2 can be drawn. The first virtual line segment VL1 is a line segment which links the gas hole GH and the specific point P by passing only through the internal space S without intersecting with the inner wall 31 of the case 30. The second virtual line segment VL2 is a line segment which links the specific point P, which is the end of the first virtual line segment VL1, and the discharge outlet 71 by passing only through the internal space S without intersecting with the inner wall 31 of the case 30.

That is, the inner wall 31 of the case 30 has a specific point P from which the first virtual line segment VL1 that links the gas hole GH and the specific point P by passing only through the internal space S and the second virtual line segment VL2 that links the specific point P and the discharge outlet 71 by passing only through the internal space S can be drawn.

In FIG. 4, only one specific point P is shown as a representative example, but the inner wall 31 has innumerable specific points P, other than the specific point P shown in FIG. 4, from which the first and second virtual line segments VL1 and VL2 defined as described above can be drawn. At least some of the innumerable specific points P are positioned on the curved surface 31A of the inner wall 31 of the case 30.

The inner wall 31 of the case 30 has a projecting portion 32. A virtual line VL3 which links the specific point P and the discharge outlet 71 with the shortest distance is drawn on the inner wall 31 of the case 30. The projecting portion 32 is positioned on the virtual line VL3. More specifically, a portion of the inner wall 31 which is at the top end of the side wall 61 and which is in the vicinity of the first end of the pipe 70 is used as the projecting portion 32. It is seen that the projecting portion 32 has a projecting shape when the virtual line VL3 is traced from the specific point P to the discharge outlet 71. It is also seen that a step is formed by the projecting portion 32 when the virtual line VL3 is traced from the specific point P to the discharge outlet 71. In the embodiment, the dimension L1 of the projecting portion 32, that is, the projecting length of the projecting portion 32, is 1.5 mm. The dimension L1 of the projecting portion 32 is a distance in the upward direction UD between the top end of the exit 66 and the end of the top wall 62 closer to the step. The virtual line VL3 is indicated by the thick line in FIG. 4.

### Operation of Embodiment

A user uses the nebulizer 10 by holding the pump case 20 with the hand, for example, and by inserting part of the nebulizer 10 including the discharge outlet 71 of the pipe 70 into the mouth. The user holds the pump case 20 so that the pump case 20 is positioned farther in the direction of gravity than the discharge outlet 71. The nebulizer 10 is used in a state in which a liquid, such as a liquid medicine, is stored in the internal space S. The liquid is thus collected on the bottom wall 51 of the tank 50 within the internal space S.

When the pump 21 is driven, air is pumped out of the pump 21 and flows through the gas supply path RG via the tube 22 and the connecting pipe 23. Air is then ejected from the gas hole GH to the internal space S.

The gas ejected from the gas hole GH passes by the vicinity of the liquid hole LH. This places the vicinity of the liquid hole LH in a negative pressure. A flow is thus generated inside the liquid supply path RL in a direction from one end to the other end. Since one end of the liquid supply path RL is located in the vicinity of the bottom wall 51 of the tank 50, the liquid stored in the tank 50 enters the inside of the liquid supply path RL. Then, the liquid is discharged from the liquid hole LH.

When the liquid discharged from the liquid hole LH is supplied to the vicinity of the gas hole GH, the gas ejected from the gas hole GH collides with the liquid. This changes the liquid into fine droplets. The fine droplets flow through the internal space S toward the discharge outlet 71 due to a flow of the gas. At this time, atomized droplets flow to the discharge outlet 71 typically along the first and second virtual line segments VL1 and VL2. Part of the atomized liquid adheres to the inner wall 31 of the case 30. The liquid attached to the inner wall 31 drops to the bottom wall 51 of the tank 50 in the internal space S by its own weight.

### Advantages of Embodiment

(1) In the above-described embodiment, the inner wall 31 of the case 30 has a specific point P from which a straight first virtual line segment VL1 that links the gas hole GH and the specific point P by passing only through the internal space S and a straight second virtual line segment VL2 that links the specific point P and the discharge outlet 71 by passing only through the internal space S can be drawn. Accordingly, a flow path of a gas from the gas hole GH to the discharge outlet 71 is curved at one portion. After the liquid is atomized and is changed to droplets, small droplets are most likely to flow to the discharge outlet 71 without colliding with the inner wall 31 of the case 30 while large droplets adhere to the inner wall 31 of the case 30.

Since the flow path of the gas is curved only at one portion, small droplets do not excessively adhere to the inner wall 31 of the case 30, which would occur if the direction of the flow path of the gas is changed multiple times. As a result, small droplets, which are sufficiently atomized, can be outputted to the outside of the case 30 at a sufficiently high flow rate while large droplets are being removed.

(2) In the above-described embodiment, large droplets which have collided with the inner wall 31 in the vicinity of the specific point P may head to the discharge outlet 71 by moving along the virtual line VL3 on the inner wall 31 in accordance with a flow of the gas within the internal space S. With the above-described configuration, the projecting portion 32 is positioned on the virtual line VL3, when the virtual line VL3, which links the specific point P and the discharge outlet 71 with the shortest distance, is drawn on the inner wall 31 of the case 30. Large droplets moving on the inner wall 31 are thus caught by the projecting portion 32 before they reach the discharge outlet 71. It is thus possible to prevent droplets attached to the inner wall 31 of the case 30 from dripping from the discharge outlet 71 of the pipe 70.

(3) In the above-described embodiment, the inner wall 31 of the case body 40 has the projecting portion 32. Droplets caught by the projecting portion 32 can thus remain in the space of the case body 40 within the internal space S and are likely to be collected somewhere downward of the space inside the case body 40.

(4) In the above-described embodiment, the pump 21 is a piezoelectric pump. While the piezoelectric pump is small and lightweight, it is relatively less powerful to pump out a gas. Hence, the ejecting force of a gas outputted from the gas hole GH is accordingly weak. Due to a weak ejecting force of the gas, relatively large droplets are likely to be generated when atomizing a liquid supplied from the liquid hole LH. Given this fact, if the pump 21 is a piezoelectric pump, the above-described advantage (1) can be exhibited more noticeably.

(5) In the above-described embodiment, the central axis CA of the pipe 70 extends in parallel with the virtual plane VP. It is thus possible to prevent droplets attached to the inner wall 31 of the case 30 from dripping from the discharge outlet 71 of the pipe 70.

(6) In the above-described embodiment, some of the innumerable specific points P are positioned on the curved surface 31A of the inner wall 31. In the vicinity of the curved surface 31A, a gas flowing in the internal space S flows in accordance with the shape of the curved surface 31A. It is thus less likely that turbulence occurs in the gas or the velocity of the gas slows down. Hence, fine droplets flowing in the vicinity of the curved surface 31A are most likely to flow to the discharge outlet 71 without adhering to the curved surface 31A.

### Other Embodiments

The above-described embodiment may be modified in the following manner and be carried out. The embodiment and the following modified examples may be combined with each other and be carried out as long as the resulting configurations do not become technically inconsistent.

The pump 21 is not limited to a piezoelectric pump. For example, the pump 21 may be a rotary pump. Depending on the type of pump 21, the pump case 20 and the connecting pipe 23 may be connected to each other with a hose, for example. In this case, the pump case 20 may be provided separately from the nebulizer 10. That is, the provision of the pump case 20 and the pump 21 for the nebulizer 10 may be omitted. If the nebulizer 10 does not include the pump case 20 and the pump 21, a user can use the nebulizer 10 by holding the tank 50, for example.

If the shape of the case 30 is such that a virtual straight line segment which links the gas hole GH and the discharge outlet 71 without intersecting with the inner wall 31 of the case 30 can be drawn, a blocking wall that blocks the flowing of droplets may be provided on this virtual straight line segment. If there is no path formed of a straight line which extends from the gas hole GH to the discharge outlet 71 by passing only through the internal space S without intersecting with another member in the internal space S, a member that eliminates the presence of such a path is not limited to the inner wall 31 of the case 30. If a blocking wall is provided as described above, it may be formed as part of the case 30 or part of the nozzle 80 or it may be formed as a member different from the case 30 or the nozzle 80.

The shape of the case 30 is not limited to the example in the embodiment. The case 30 may be formed in any shape if the inner wall 31 of the case 30 has at least one specific point P from which the first virtual line segment VL1 that links the gas hole GH and the specific point P by passing only through the internal space S and the second virtual line segment VL2 that links the specific point P and the discharge outlet 71 by passing only through the internal space S can be drawn. Additionally, the specific point P may be located at a position other than on the curved surface 31A of the inner wall 31 of the case 30. More specifically, the provision of the curved surface 31A on the inner wall 31 of the case 30 may be omitted.

If the inner wall 31 of the case 30 has multiple specific points P, the projecting portion 32 of the case 30 may be positioned on the virtual line VL3 linked to one of the multiple specific points P. In this case, the effect of catching droplets moving on this virtual line VL3 can be obtained. If a step is found when the virtual line VL3 is traced from the specific point P to the discharge outlet 71, it can be said that the inner wall 31 includes a projecting portion 32. As in the above-described embodiment, if the inner wall of the pipe 70, which is part of the virtual line VL3 including an end thereof, protrudes from the inner wall of the cover 60, it can also be said that the inner wall 31 includes a projecting portion 32.

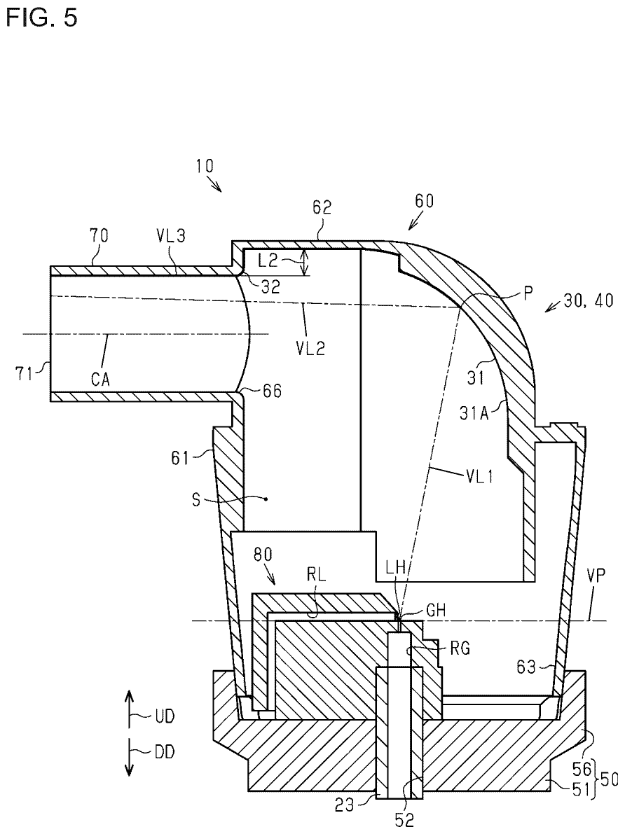

The dimension of the projecting portion 32 of the case 30 is not limited to the example in the embodiment. In a modified example shown in FIG. 5, a dimension L2 of the projecting portion 32 of the case 30 is 3.5 mm. If the dimension L2 of the projecting portion 32 is 3.5 mm, the probability of large droplets being caught is greater than that when the dimension L1 of the projecting portion 32 is 1.5 mm as in the above-described embodiment. That is, the average particle size of droplets discharged from the discharge outlet 71 becomes smaller. On the other hand, when the dimension L1 of the projecting portion 32 of the case 30 is 1.5 mm as in the above-described embodiment, the total number of droplets, that is, the number of atomized droplets, discharged from the discharge outlet 71 is greater than that when the dimension L2 of the projecting portion 32 is 3.5 mm as in the modified example in FIG. 5. If the dimension of the projecting portion 32 of the case 30 is 1.5 to 3.5 mm, a good balance between the decreased average particle size of droplets discharged from the discharge outlet 71 and the total number of droplets discharged from the discharge outlet 71 is likely to be maintained.

The dimension of the projecting portion 32 of the case 30 may be smaller than 1.5 mm or may be larger than 3.5 mm. The projecting portion 32 of the case 30 may be disposed in a space within the pipe 70. The provision of the projecting portion 32 in the case 30 may be omitted.

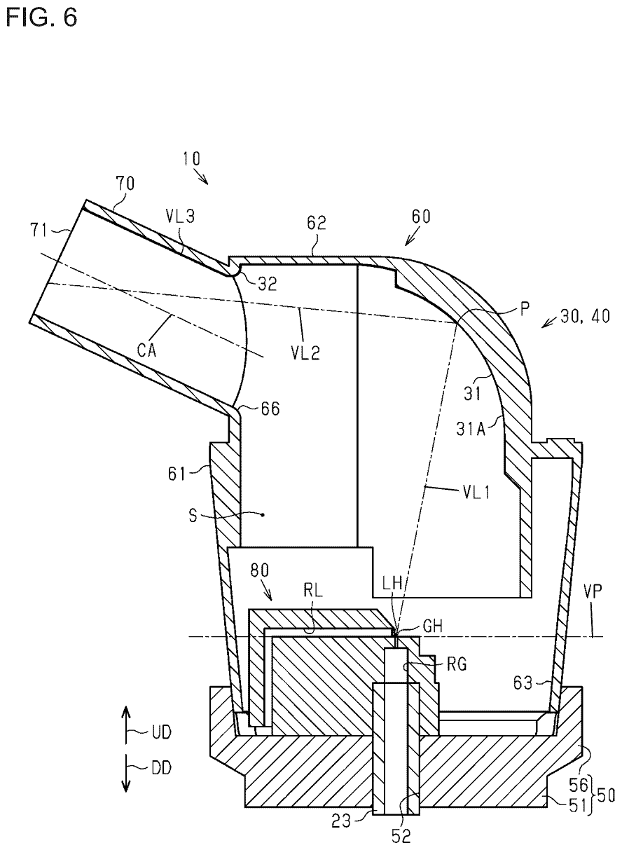

It is not essential that the extending direction of the pipe 70 is parallel with the virtual plane VP. In one example, in a modified example shown in FIG. 6, the central axis CA of the pipe 70 is inclined in the upward direction UD such that the pipe 70 is positioned farther in the upward direction UD as it becomes closer to the discharge outlet 71. In this case, too, it is possible to prevent droplets attached to the inner wall 31 of the case 30 from dripping from the discharge outlet 71 of the pipe 70.

In another example, the central axis CA of the pipe 70 may be inclined in the downward direction DD such that the pipe 70 is positioned farther in the downward direction DD as it becomes closer to the discharge outlet 71. It is not essential that the pipe 70 extends straight. For example, the pipe 70 may be curved or may extend so that the sectional area of a flow path is varied. The pipe 70 may be a mask which covers the mouth and the nose of a user. The pipe 70 may be flexible. The provision of the pipe 70 may be omitted. In this case, the exit 66 of the case body 40 serves as the discharge outlet of the case 30.

The shape of the nozzle 80 is not limited to the example in the embodiment. The shape of the nozzle 80 may be changed suitably in accordance with the shape of the case 30, for example, if the nozzle 80 has the gas hole GH and the liquid hole LH.
10 nebulizer
20 pump case
30 case
31 inner wall
32 projecting portion
40 case body
50 tank
60 cover
70 pipe
71 discharge outlet
80 nozzle
GH gas hole
LH liquid hole
P specific point
VL1 first virtual line segment
VL2 second virtual line segment

1. A nebulizer comprising:
a case having an inner wall, the inner wall defining an internal space; and
a nozzle located in the internal space and configured to atomize a liquid stored in the internal space, wherein
the case has a discharge outlet for guiding the atomized liquid to outside of the case,
the nozzle has a gas hole and a liquid hole, the gas hole being configured to eject a gas, the liquid hole being located adjacent to the gas hole and being configured to eject the liquid,
in the internal space, the case does not have any straight-line path extending from the gas hole to the discharge outlet by passing only through the internal space,
when a point is specified on the inner wall of the case and is set to a specific point, the inner wall of the case has at least one specific point specified on the inner wall, the specific point being a point from which a virtual line segment linking the gas hole and the specific point by passing only through the internal space and a virtual line segment linking the specific point and the discharge outlet by passing only through the internal space are drawn.

2. The nebulizer according to claim 1, wherein:
the inner wall of the case includes a projecting portion; and
when a virtual line linking the specific point and the discharge outlet with a shortest distance is drawn on the inner wall of the case, the projecting portion is positioned on the virtual line.

3. The nebulizer according to claim 2, wherein:
the case includes a case body and a pipe, the case body housing the nozzle therein, the pipe having a tubular shape, a first end of the pipe being linked with the case body, a second end of the pipe being used as the discharge outlet; and
an inner wall of the case body includes the projecting portion.

4. The nebulizer according to claim 1, further comprising:
a piezoelectric pump configured to output the gas.

5. The nebulizer according to claim 1, wherein:
the case includes a case body and a pipe, the case body housing the nozzle therein, the pipe having a tubular shape, a first end of the pipe being linked with the case body, a second end of the pipe being used as the discharge outlet;
when a pointing direction of the gas hole is set to a first direction and a virtual plane perpendicular to the first direction is imagined, a central axis of the pipe is parallel with the virtual plane or is inclined in the first direction such that the pipe is positioned farther in the first direction as the pipe becomes closer to the discharge outlet.

6. The nebulizer according to claim 2, further comprising:
a piezoelectric pump configured to output the gas.

7. The nebulizer according to claim 3, further comprising:
a piezoelectric pump configured to output the gas.

8. The nebulizer according to claim 2, wherein:
the case includes a case body and a pipe, the case body housing the nozzle therein, the pipe having a tubular shape, a first end of the pipe being linked with the case body, a second end of the pipe being used as the discharge outlet;
when a pointing direction of the gas hole is set to a first direction and a virtual plane perpendicular to the first direction is imagined, a central axis of the pipe is parallel with the virtual plane or is inclined in the first direction such that the pipe is positioned farther in the first direction as the pipe becomes closer to the discharge outlet.

9. The nebulizer according to claim 3, wherein:
the case includes a case body and a pipe, the case body housing the nozzle therein, the pipe having a tubular shape, a first end of the pipe being linked with the case body, a second end of the pipe being used as the discharge outlet;
when a pointing direction of the gas hole is set to a first direction and a virtual plane perpendicular to the first direction is imagined, a central axis of the pipe is parallel with the virtual plane or is inclined in the first direction such that the pipe is positioned farther in the first direction as the pipe becomes closer to the discharge outlet.

10. The nebulizer according to claim 4, wherein:
the case includes a case body and a pipe, the case body housing the nozzle therein, the pipe having a tubular shape, a first end of the pipe being linked with the case body, a second end of the pipe being used as the discharge outlet;
when a pointing direction of the gas hole is set to a first direction and a virtual plane perpendicular to the first direction is imagined, a central axis of the pipe is parallel with the virtual plane or is inclined in the first direction such that the pipe is positioned farther in the first direction as the pipe becomes closer to the discharge outlet.
